(19) United States
(12) Patent Application Publication
(10) Pub. No.: US 2004/0176746 A1
(43) Pub. Date: Sep. 9, 2004

(54) Urine collection system for males utilizing a flexible external catheter and vacuum assistance

(76) Inventor: Arthur Esguerra Forral, San Antonio, TX (US)

Correspondence Address: Arthur E. Forral, 5806 Woodridge Oaks, San Antonio, TX, 78249, US

(21) Appl. No.: 10/376,558

(22) Filed: Mar. 3, 2003

(51) Int. Cl. A61M 27/00

(52) U.S. Cl. 604/544

(57) ABSTRACT

An improved urine collection system for males comprising an elastic waistband (12), a sheath support member (14), a flexible removable and replaceable catheter assembly (20), means to releasably secure the flexible external catheter assembly (20) to the sheath support member (14), a substantially rigid urine receptacle assembly (40), means to induce a partial vacuum, a first tubing (37) providing fluid communication between the catheter assembly (20) and the urine receptacle assembly (40), and a second tubing (52) providing vacuum communication between the vacuum means and the urine receptacle assembly (40).

### FEDERALLY SPONSORED RESEARCH

Not applicable

### SEQUENCE LISTING OR PROGRAM

Not applicable

### BACKGROUND

1. Field of Invention

This invention relates to a male urine collection system, specifically a system utilizing a flexible external catheter and vacuum assistance.

2. Description of Prior Art

Military fighter pilots desire an effective urine management device they can use while seated inside a cockpit. The most widespread method for male military pilots is to urinate directly into a closable bag. The problem with this method is the pilot has to accomplish several tasks that impair his ability to control his aircraft. For example, a pilot typically removes his gloves, puts slack in his seat restraints, shifts himself forward on the ejection seat, unzips his flight suit, exposes his penis, opens and holds the closable bag, urinates, closes the bag, stows the bag, zips up his flight suit, shifts back onto his flight seat, tightens up his seat restraints, and puts his gloves back on. These actions impair the pilot's control over his aircraft because both hands may be off the flight controls, and his attention and vision will be focused on non-flying matters. A mishap could occur. Another potential problem is that if the cockpit is contaminated with chemical or biological agents, the pilot risks exposure to these agents during urination. Further, the pilot could accidentally drop the receptacle during urination resulting in spillage inside the cockpit. Urine is corrosive to aircraft metals and will adversely effect avionics equipment. An effective but highly unpopular urine management alternative is the insertion of an internal catheter in the pilot's urethra. The catheter is connected to a tube that drains into a urine storage receptacle. Most pilots avoid this method due to the discomfort and the inconvenience of seeking medical personnel to have the internal catheter inserted and later removed.

Male fighter pilots desire a simple to use, noninvasive urine collection system. There are numerous portable prior art systems designed for incontinent males that may have application, but a closer examination shows their limitations. One prior art approach is a system that consists of an external and flexible catheter worn over the penis, a urine collection receptacle, and a drain tube providing fluid communication between the two. These systems typically rely on gravity to cause urine to flow towards the urine collection receptacle. The drain tube must be positioned below the catheter, and the urine collection receptacle must be positioned as the lowest item in the system. This order is necessary to maximize urine storage and reduce backflow problems. When the male urinates, urine flows out of the catheter, into the drain tube, and into the collection receptacle. U.S. Pat No. 20020193763 to Kulikov (2002), U.S. Pat. No. 5,346,483 to Thaxton (1994), U.S. Pat. No. 4,713,067 to Rothenberg et al. (1987), and U.S. Pat. No. 4,073,295 to Laufbahn (1978) are examples of gravity-dependent systems. Since the collection receptacle has to be the lowest item in the system and no part of the drain tube can be at the highest point in the system, there are limitations on how and where these systems can be used.

Fighter cockpits are very cramped and there generally is not any room immediately down and to the left, right, or back of the pilot. Therefore the aforementioned gravity-dependent systems require that the collection receptacle and drain tube be located down and in front of the pilot relative to the catheter. One embodiment is for the wearer to don the catheter and position the drain tube and a flexible collection receptacle along a leg inside the wearer's trousers. However, these devices would be ineffective for fighter pilots because they would not be compatible with antigravity trousers pilots wear over their flight suit. Antigravity trousers are an important part of the fighter pilot's life support equipment. These trousers apply pressure to the legs during high g maneuvers. When these trousers are donned, they are fitted snug to the legs and they cannot accommodate the aforementioned patent devices. Flexible urine collection receptacles would not be free to expand, and drain tubing may become kinked resulting in reduced flow and backflow problems. Putting a rigid container in place of the flexible urine collection receptacle would not be satisfactory either because it will reduce the antigravity suit's effectiveness and cause discomfort.

Another embodiment of these gravity-dependent systems is to route the drain tube outside the pants, either out through an open fly or out the bottom of a pant leg, and position the urine collection receptacle down and in front of the pilot. However, this set up will create significant problems since the drain tube and collection receptacle will interfere with the pilot's access to foot controls and possibly other flight controls. This set up could also create an uncomfortable situation for the pilot since these items take up foot well space in an already cramped cockpit. Also, since the pilot will have poor visual contact with much of the system, he could accidentally step on the collection receptacle and drain tube and cause the system to leak.

A common problem with many prior art catheters is that they quickly become uncomfortable to wear because catheters are typically constructed of impermeable materials that tend to entrap vapor and moisture from perspiration and residual urine. Body heat may also be entrapped inside the catheter. This condition can lead to severe irritation and even skin damage if the catheter is worn for long periods. Air circulation would greatly improve comfort but this is difficult to achieve in catheters that are closely fitted to the penis like a condom. U.S. Pat. No. 5,009,649 to Goulter et al. (1991), U.S. Pat. No. 4,840,625 to Bell (1989), and U.S. Pat. No. 3,032,038 to Swinn (1962) are examples of devices with condom catheters. Even loosely fitting external catheters as in U.S. Pat. No. 4,997,427 to Bowen (1991), U.S. Pat. No. 4,994,051 to Walsh (1991), and U.S. Pat. No. 4,713,067 to Rothenberg et al. (1987) will entrap vapor, moisture, and body heat since there is no mechanism to promote active aeration of the catheter.

Another prior art approach to urine management is to suction pump the pilot's urine to a more suitable storage location. U.S. Pat. No. 5,002,541 to Conkling et al. (1991), U.S. Pat. No. 4,531,939 to Kimura et al. (1982), and U.S. Pat. No. 4,281,655 to Terauchi (1981) are examples of devices using an electromechanical pump to draw urine from a housing that interfaces with the penis. Instead of using flexible catheters, like the ones cited in the patents of the previous paragraphs, these devices use substantially rigid housings. Substantially rigid housings are used because a flexible catheter will collapse around the penis in the presence of a suction means. A catheter that collapses in this manner would be uncomfortable to the wearer because it can apply intense pressure to the male organ, and it will entrap residual moisture and urine against the skin. Therefore, few prior art devices use suction means in conjunction with flexible catheters.

These electromechanical systems have drawbacks when used aboard fighter aircraft. For one, the aircraft would have to be modified to incorporate this device. This modification would be expensive, adds weight, and requires maintenance. Another reason is that many of these prior art devices use housings that are cumbersome to operate in a cockpit. Further, the pilot may have to contend with stowing items after use.

Another problem with a majority of prior art flexible catheters and housings are that they take considerable manufacturing efforts to make and are therefore cost prohibitive to throw away. This creates at least three problems for a user. First, users generally do not want to take the time nor the trouble to clean and maintain these types of items. Second, despite the best cleaning efforts, these items often retain a terrible odor and discoloration since urine will permeate into the plastics and rubbers typically used to make these devices. Third, the user has to deal with storing these unsanitary devices when not in use.

U.S. Pat. No. 5,318,550 to Cermak (1994) solves some of the problems with gravity-dependent and electromechanical urine collection systems. In that device, a pump bulb is placed between a condom catheter and a urine collection bag. When the wearer urinates, he pumps the pump bulb, which forces urine into the collection bag. However, there are at least three drawbacks with this device. First, it has the problems associated with condom catheters in that air circulation is limited. In order for air to flow in this device, the catheter must first be collapsed around the penis. When the user repeatedly pumps the pump bulb, it exerts suction at the tip of the catheter that eventually causes it to collapse around the penis. Replenishing air is allowed to enter through a valve at the tip of the catheter to relieve the suction resulting in some expansion of the catheter. Thus, the aeration cycle involves the volumetric collapse and expansion of the catheter about the penis. However, this method is ineffective for drawing in a good flow of air because a condom catheter will not expand significantly about the penis when the suction is relieved, thus little air will be drawn back into the catheter, especially if the penis occupies a significant portion of the catheter. A second drawback to this system is that continually collapsing the catheter about the penis to promote aeration may be uncomfortable to wearer. A third drawback is that the pump bulb will come in contact with urine meaning that cleaning of the bulb would have to occur after use.

Ultimately, the majority of fighter pilots have resigned themselves to using a closable bag for urine management. However, pilots would like a better solution. Accordingly, several objects and advantages of my invention are:

(a) to significantly reduce the number of actions a male fighter pilot needs to take in order to relieve himself thereby enhancing his ability to control his aircraft;

(b) to overcome the limitations imposed by gravity-dependent urine management systems;

(c) to provide a comfortable and reliably attached bodily worn urine relief item fully compatible with a fighter pilot's clothing and life support equipment, including chemical-biological protective clothing;

(d) to provide a system compatible with all cockpits, one that does not interfere with the pilot nor requires expensive aircraft modifications to aircraft;

(e) to effectively alleviate heat, vapor and moisture entrapment common in external catheters;

(f) to provide a cost effective means to eliminate the need to clean and maintain external catheters;

(g) to provide reliable containment of urine to prevent damage to aircraft metals and avionics systems;

A further object and advantage is to provide a new approach to urine management that can be used by any male confined to a seated position.

### SUMMARY

In accordance with the present invention a urine collection system for males comprising a waistband, a sheath support member, a flexible removable and replaceable external catheter assembly, means to releasably secure the flexible external catheter assembly to the sheath support member, a substantially rigid urine receptacle assembly, means to induce a partial vacuum, tubing providing fluid communication between the catheter assembly and the urine receptacle assembly, and tubing providing vacuum communication between the vacuum means and the urine collection receptacle.

### DRAWINGS

Drawing Figures

FIG. 1 shows a complete urine collection system including a catheter garment assembly, a urine receptacle assembly, a vacuum aspirator bulb assembly, and a pair of tubes proving fluid and vacuum communication.

FIG. 2 shows a complete urine collection system in relationship to a seated pilot.

FIG. 3 shows all the parts of a removable and replaceable catheter sheath assembly, and an elastic waistband, a sheath support member, and a pair of elastic straps.

FIG. 4 shows a catheter drain tip.

FIGS. 5A to 5C show various aspects of a vacuum aspirator bulb assembly.

FIG. 6 shows a complete urine collection system with a catheter garment assembly worn underneath a pilot's flight suit.

### REFERENCE NUMERALS IN DRAWINGS

| | |
|---|---|
| 10 | male urine collection system |
| 11 | external catheter garment assembly |
| 12 | elastic waistband |
| 14 | sheath support member |
| 15 | first aperture |
| 17 | first attachment assembly |
| 18 | second attachment assembly |
| 19 | plurality of attachment tabs |
| 20 | flexible removable and replaceable catheter assembly |
| 21 | tapered extension portion |
| 22 | elastic straps |
| 23 | penile sheath body |
| 25 | second aperture |
| 27 | vacuum relief hole |
| 28 | plurality of snap connector fasteners |
| 29 | plurality of complementary snap connector fasteners |
| 30 | first closable union fitting |
| 31 | first open or close lever |
| 33 | catheter drain tip |
| 37 | first tubing |
| 40 | urine receptacle assembly |
| 42 | substantially rigid container |
| 44 | drain cap |
| 46 | inlet port fitting |
| 48 | vacuum port |
| 50 | second closeable union fitting |
| 51 | second open or close lever |
| 52 | second tubing |
| 60 | vacuum aspirator bulb assembly |
| 62 | aspirator bulb body |
| 64 | first one-way valve |
| 65 | valve fitting |
| 67 | second one-way valve |
| 90 | third tubing |
| 92 | union fitting |
| 100 | retaining strap |
| 110 | tape strip |

### DETAILED DESCRIPTION

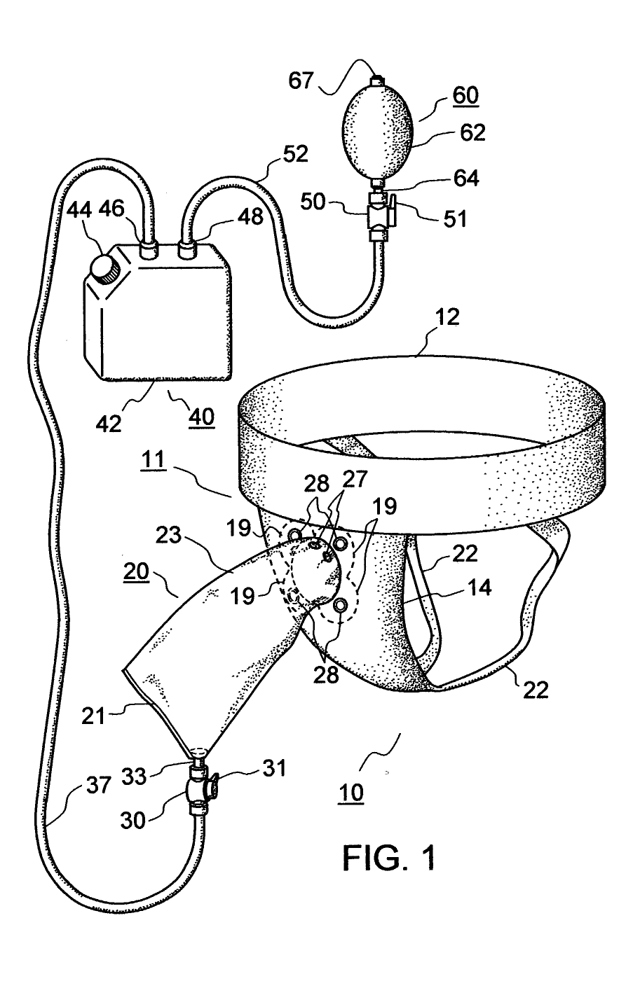

### DESCRIPTION—FIGS. 1-5C—PREFERRED EMBODIMENT

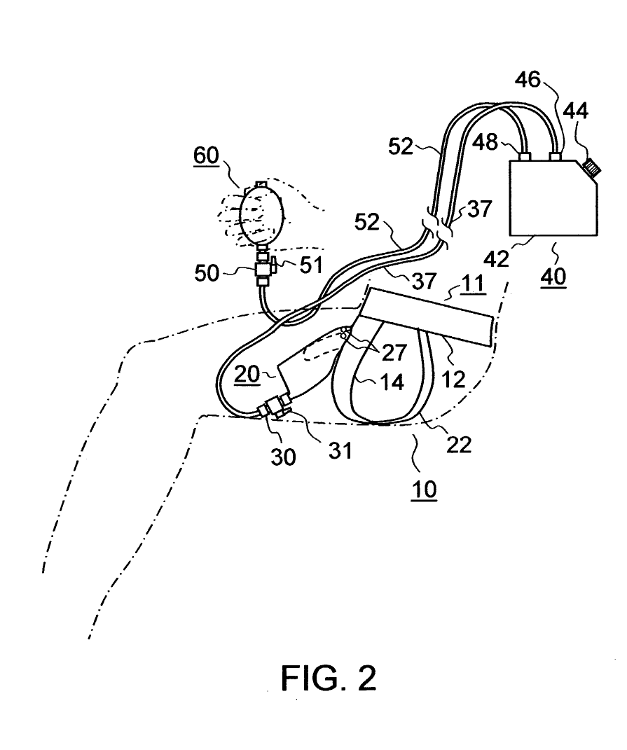

A preferred embodiment of the male urine collection system featuring a flexible external catheter and vacuum assistance is illustrated in FIGS. 1 and 2, designated by reference numeral 10. The male urine collection system 10 includes an external catheter garment assembly 11. The external catheter garment assembly 11 includes an elastic waistband 12, a sheath support member 14 attached to the waistband 12, a flexible removable and replaceable catheter assembly 20, and elastic straps 22. Sheath support member 14 has a first aperture 15 through which catheter assembly 20 is inserted. Sheath support member 14 includes a first attachment assembly 17 positioned circumferentially around the aperture 15. First attachment assembly 17 includes a plurality of snap connector fasteners 28 as its attachment means. The fastening points of snap connector fasteners 28 are located on the interior of sheath support member 14. A pair of elastic straps 22 connect waistband 12 to sheath support member 14. Elastic straps 22 stabilizes sheath support member 14 to waistband 12. Sheath support member 14 can be made from a comfortable washable textile. Waistband 12 and elastic straps 22 can be made from comfortable elastic and washable textiles. The waistband 12, sheath support member 14 and elastic straps 22 can be sewn together to form a unified structure.

Catheter assembly 20 includes a penile sheath body 23 that loosely over fits the penis. Catheter assembly 20 also includes a second attachment assembly 18, which is complementary to the first attachment assembly 17 and is attachable thereto. Catheter assembly 20 also includes a second aperture 25 through which a penis is inserted. Catheter assembly 20 also includes a tapered extension portion 21 connected to the penile sheath body 23. Tapered extension portion 21 is also situated as the lowest part of catheter assembly 20. Catheter assembly 20 also includes a substantial number of vacuum relief holes 27 located adjacent to the second attachment assembly 18 and arranged near the top of penile sheath body 23. The tapered extension portion 21 includes a catheter drain tip 33 that allows the outflow of fluids and air from the catheter assembly.

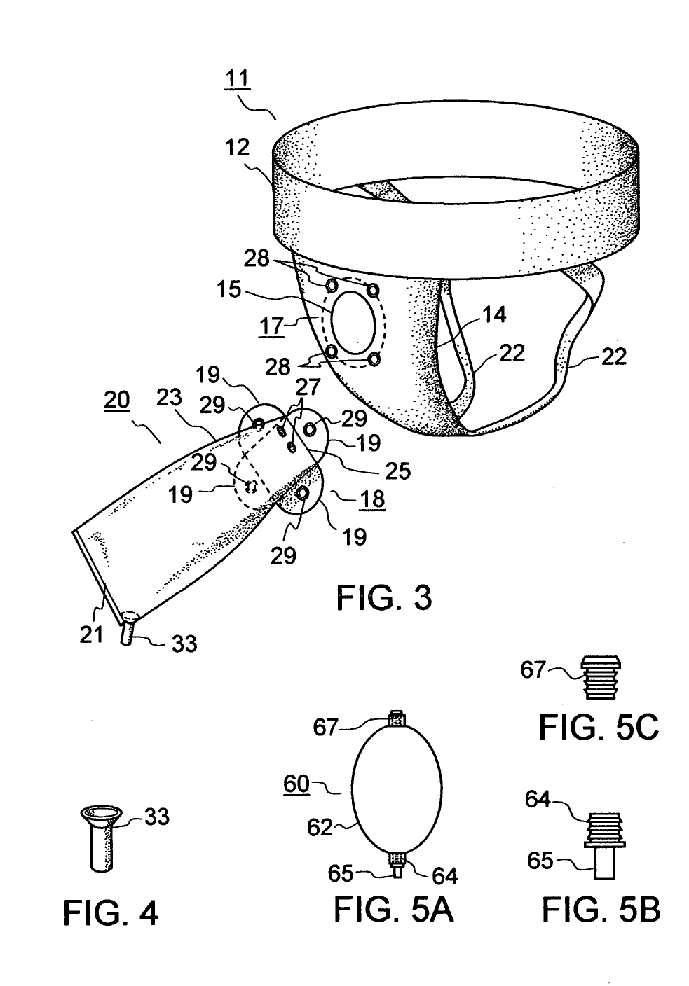

The penile sheath body 23, the second aperture 25, the second attachment assembly 18, and the tapered extension portion of catheter assembly 20 can be formed from a single sleeve of flexible and substantially resilient material sealed at one end (FIG. 3). The second attachment assembly 18 is formed into a plurality of attachment tabs 19 and includes a plurality of complementary snap connector fasteners 29 that are complementary to the plurality of snap connectors 28. The circumference of second aperture 25 is sized larger than the circumference of the first aperture 15 so that when catheter assembly 20 is inserted into aperture 15 and fastened to first attachment assembly 17, penile sheath body 23 will bunch up slightly at first aperture 15 (FIG. 1). When worn under a flight suit, the sleeve material should not readily melt and adhere to the skin when the pilot is exposed to a short duration of intense heat. Many rubber and some plastic materials are suitable.

Catheter drain tip 33 is dimensioned so that it may be easily inserted into, or withdrawn from, one end of a first closable union fitting 30. Closable union fitting 30 includes a first open or close lever 31. The other end of union fitting 30 is connected to a urine receptacle assembly 40 through a first tubing 37. Various manufacturers make suitable closable union fittings 30, typically with a feature that allows quick insertion or removal of tubing. Inexpensive, small-diameter vinyl tubing can be used for first tubing 37.

The urine receptacle assembly 40 includes a substantially rigid container 42, a drain cap 44 and an inlet port fitting 46 located at the top of substantially rigid container 42. The urine receptacle assembly 40 connects to first tubing 37 through inlet port fitting 46. Urine receptacle assembly 40 also includes a vacuum port 48 located at the top of substantially rigid container 42. High-density polyethylene bottles of various sizes will work because of their shatter-resistance, low weight, and adequate rigidity.

A second tubing 52 connects vacuum port 48 to a second closable union fitting 50. Closable union fitting 50 includes a second open or close lever 51. Second closable union fitting 50 also connects to a vacuum aspirator bulb assembly 60. As with first closable union fitting 30, second closable union fitting 50 is available from various manufacturers. Small-diameter vinyl tubing can be used for second tubing 52.

Vacuum aspirator bulb assembly 60 includes an aspirator bulb body 62, a first one-way valve 64, and a second one-way valve 67. First one-way vale 64 includes a valve fitting 65. Valve fitting 65 is dimensioned so that it may be easily inserted into, or withdrawn from, second closable union fitting 50. First one-way valve 64 and second one-way valve 67 are oriented in aspirator bulb body 62 so that a partial vacuum occurs momentarily at the tip of valve fitting 65 every time the aspirator bulb body 62 is squeezed and released. The aspirator bulb body 62 is made of a substantially resilient elastic material so that the aspirator bulb body 62 will return to its original shape after it is released.

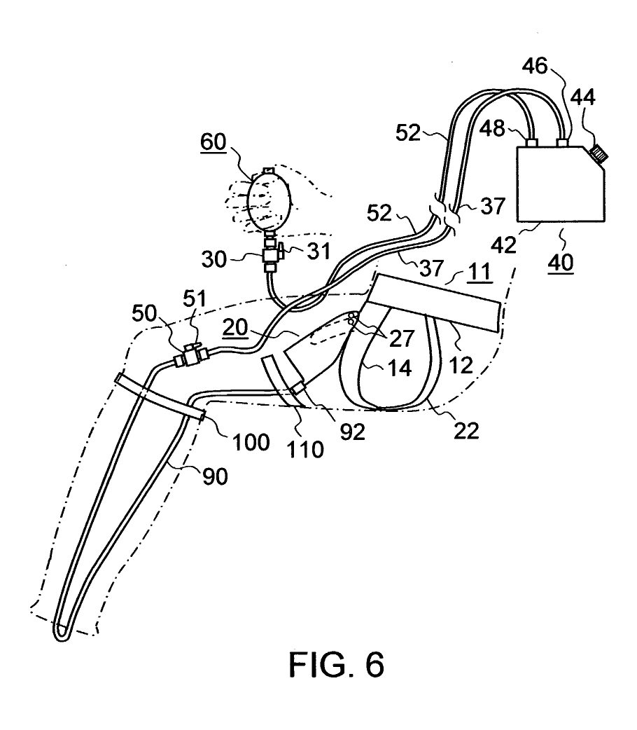

### FIG. 6—ADDITIONAL EMBODIMENT

A second embodiment is shown in FIG. 6. The second embodiment is as in the preferred embodiment above except catheter drain tip 33 is connected to a third tubing 90 sized long enough to route down and out a pant leg of the pilot's flight suit, and up to first closable union fitting 30. FIG. 6 depicts third tubing 90 routing down, out, and up the right inside pant leg. The second embodiment further includes a union fitting 92 that joins third tubing 90 to catheter drain tip 33. This embodiment further includes a retaining strap 100 that is used to keep third tubing 90 within arms reach of the pilot. This embodiment further includes a tape strip 110 used to keep catheter assembly 20 positioned on an inside thigh. The second embodiment can be used with normal flight garments, but its best application would be when it is used underneath chemical-biological protective clothing in a chemical warfare environment.

Advantages

From the description above, a number of advantages of my male external catheter system with vacuum assistance become evident:

(a) The system provides a new approach to urine management that will make it safer, easier, and more convenient for pilots to urinate while seated in the confined cockpit of an airborne fighter aircraft.

(b) The vacuum assist feature allows the urine collection receptacle to be placed in any out of the way location in a cockpit regardless of height relative to the catheter; this location may include mounting the urine collection receptacle on the pilot.

(c) Tubing routes in the cockpit are not limited by tube lengths or tube position relative to the catheter; thus, interference with the pilot can be eliminated by carefully routing the tubing paths.

(d) The bodily worn items can be made comfortable, secure, and fully compatible with the pilot's clothing and life support gear; with minor adaptation, they can also be compatible with chemical-biological protective clothing.

(e) The vacuum aspirator bulb provides increased comfort by removing excess heat, vapor, and moisture from the catheter.

(f) The catheter can be easily constructed from very cheap materials making it ideal to throw away after use.

(g) The vacuum aspirator is very effective in draining urine from the catheter greatly reducing the possibility of urine backflow onto the penis.

Operation—FIGS. 1, 2, 6

Prior to flight operations the urine receptacle assembly 40 is secured in an upright position in any available out-of-the-way location inside the cockpit. A deodorizing fluid may be placed in urine receptacle assembly 40 to control urine odor. First tubing 37 is installed between inlet port fitting 46 and first closable union fitting 30. First tubing 37 is sized and positioned so that first closable union fitting 30 is in within easy reach of a seated pilot. First tubing 37 is also routed so that it and union fitting 30 does not interfere with the pilot. Second tubing 52 and second closable union fitting 50 are similarly sized and installed. At the pilot's option, vacuum aspirator bulb assembly 60 may also be connected to second closable union fitting 50 prior to flight. During his flight preparation, the pilot dons the external male catheter garment assembly 11. The pilot puts on his flight suit with catheter assembly 20 tucked inside the suit. The pilot dons all other life support gear and performs his flight mission.

When the pilot is ready to urinate during flight, he unzips his flight suit at the crotch and extends catheter assembly 20 out of his fly (FIG. 2). He inserts catheter drain tip 33 into the first closable union fitting 30. If not already installed, the pilot inserts valve fitting 65 of the vacuum aspirator bulb assembly 60 into the second closable union fitting 50. The pilot opens first closable union fitting 30 and second closable union fitting 50 by placing first open or close lever 31 and second open or close lever 51 in the open positions. The pilot should be able to accomplish each of these tasks very quickly.

The pilot then urinates into catheter assembly 20 while pumping vacuum aspirator bulb body 62 with one hand. Pumping the aspirator bulb body 62 induces partial vacuum pressure in urine receptacle assembly 40 through second tubing 52. This partial vacuum is communicated to catheter assembly 20 through first tubing 37. This partial vacuum causes any fluid in the tapered extension portion 21 to flow through first tubing 37 and into urine receptacle assembly 40. Vacuum sealing of catheter assembly 20 around the penis is prevented by vacuum relief holes 27. These holes also allow a flow of air into the catheter assembly 20 equal in volume to the air pumped out of the aspirator bulb body 62 after it is released.

The pilot continues pumping the aspirator bulb body 62 until catheter assembly 20 is empty. The pilot closes first and second closable union fittings 30 and 50, respectively, by placing first open or close lever 31 and second open or close lever 51 in the closed positions. This action keeps the urine reliably contained. The pilot then disconnects catheter drain tip 33 from first closeable union fitting 30, tucks catheter assembly 20 back into his flight suit, and zips up the suit. The pilot may remove vacuum aspirator bulb assembly 60 from second closable union fitting 50 and stow it, or he may keep it in place. The pilot can repeat the process whenever he needs to urinate. He can also repeat this process if there is excess heat and moisture build up inside catheter assembly 20 and aeration is needed for comfort.

For the second embodiment (FIG. 6), the external catheter garment assembly 11 stays underneath the pilot's clothing. When the pilot prepares for flight, he connects catheter drain tip 33 to third tubing 90 with union fitting 92. Third tubing 90 is routed down one pant leg, out the bottom of that pant leg, and over the antigravity trousers. Retaining strap 100 is used to keep the open end of third tubing 90 in place near the knee. When the pilot is ready to urinate during flight, he inserts third tubing 90 into first closable union fitting 30. He places first open or close lever 31 and second open or close lever 51 to the open positions. The pilot urinates into catheter assembly 20 while pumping vacuum aspirator bulb body 62. When finished, he places first open or close lever 31 and second open or close lever 51 in the closed positions. He then disconnects third tubing 90 from first closable union fitting 30. This second embodiment guards the pilot against exposure to chemical-biological agents that may be in the cockpit. Third tubing 90 is made from compliant, small diameter tubing that will not reduce the effectiveness of antigravity trousers. Vinyl or latex tubing is suitable.

After finishing his flight, the pilot disposes of catheter assembly 20 and all tubing that has come in contact with urine. All other items that have come in contact with urine are washed for reuse. Normally, items to be washed are the urine receptacle assembly 40, first closable union fitting 30 and second closable union fitting 50. The elastic waistband 12, sheath support member 14, and elastic straps 22 may be laundered as normal clothing.

Conclusions, Ramifications, and Scope

Accordingly, the reader will see that system features of this invention can be used to bring about added and great improvements in urine relief management for male pilots seated in small and confined cockpits in that

it provides a quick, easy, and convenient way for a male pilot to relieve himself while seated in a small cockpit, thereby enhancing his ability to keep his aircraft under control while urinating;

it allows the urine storage receptacle to be secured in an out-of-the-way location inside any cockpit; this eliminates interference problems and eliminates the possibility of damage to aircraft metals and avionics as a result of mishandling the urine container;

it allows far more drain tubing routing options which eliminates interference problems when compared to gravity dependent systems;

it provides comfortably and reliably attached bodily-worn urine relief items compatible with a fighter pilot's clothing and life support equipment, including chemical-biological protective clothing;

it provides increased comfort capability with its ability to draw out excess heat, vapor and moisture from the external catheters;

it provides reduced system care and maintenance requirements on the part of the user through its emphasis on utilizing low cost and easily manufactured items that may be thrown away after use;

it provides an increased urine drainage rate from catheters sheath, compared to a comparable gravity dependent system, reducing the possibility of urine backflow.

Although the description above contains many specificities, these should not be construed as limiting the scope of the invention but as merely providing illustrations of some of the presently preferred embodiments of this invention. For example, the catheter assembly can have various shapes and sizes. Also, the vacuum relief holes can have one-way valves that allow air in when a vacuum is applied and close when the vacuum is stopped to keep urine contained. Another example is placing a filter between the vacuum port and the vacuum aspirator bulb assembly to prevent urine moisture and odors from reaching and exiting the vacuum aspirator bulb. Another example is the urine collection receptacle can be of many different shapes and sizes.

Thus the scope of the invention should be determined by the appended claims and their legal equivalents, rather than by the examples given.

I claim:

1. A urine collection system for males utilizing a flexible external catheter and vacuum assistance comprising:
a. a waistband
b. a sheath support member attached to said waistband, said sheath support member including a first aperture, said sheath support member also including a first attachment assembly positioned circumferentially around said first aperture,
c. a flexible removable and replaceable catheter assembly, said catheter assembly including a penile sheath body sized loosely to overfit the penis, said catheter assembly including a second aperture through which a penis is inserted, said catheter assembly including a second attachment assembly complementary to said first attachment assembly, said penile sheath body including a substantial number of vacuum relief holes adjacent to said second attachment assembly, said catheter assembly including a tapered extension portion connected to said penile sheath body, said tapered extension portion including a drain tip for draining urine from said catheter assembly,
d. means to releasably secure said first attachment assembly to said second attachment assembly,
e. a substantially rigid urine receptacle assembly, said receptacle assembly including an inlet port in fluid communication with said tapered extension portion of said penile sheath, said receptacle assembly including a vacuum port located at the top of said receptacle assembly, and
f. means to induce a partial vacuum at said vacuum port of said receptacle assembly.

2. The apparatus of claim 1 wherein said means to induce a partial vacuum is provided by a vacuum aspirator bulb assembly.

3. The apparatus of claim 1 further including a pair of straps, connected between said waistband and said sheath support member, which stabilizes said sheath support member with said waistband.

4. The apparatus of claim 1 wherein said penile sheath body, said second aperture, said second attachment assembly, and said tapered extension portion are together formed from a sleeve of flexible and liquid impermeable material, said second attachment assembly formed into a plurality of attachment tabs positioned circumferentially around said second aperture, said tapered extension portion formed by sealing an end opposite of said second aperture of said sleeve, said penile sheath body including said substantial number of vacuum relief holes adjacent to said attachment tabs.
